(19) United States
(12) Patent Application Publication
(10) Pub. No.: US 2024/0053284 A1
(43) Pub. Date: Feb. 15, 2024

(54) System and Method for Tomographic Imaging of Core Samples

(71) Applicant: Orimtech Ltd., Buffalo Grove, IL (US)

(72) Inventors: Boris S. Goldberg, Buffalo Grove, IL (US); Serguei Gouzeev, Discovery Bay, CA (US)

(21) Appl. No.: 18/447,555

(22) Filed: Aug. 10, 2023

Related U.S. Application Data
(60) Provisional application No. 63/371,158, filed on Aug. 11, 2022.

(51) Int. Cl. G01N 23/046; G01N 33/24; G01N 23/083

(52) U.S. Cl. CPC G01N 23/046; G01N 33/24; G01N 23/083; G01N 2223/3303; G01N 2223/316; G01N 2223/616

(57) ABSTRACT

A CT scanning system having an industrial robot with X-Ray source and detector assembly as a tool to perform CT scanning of geological formation samples and such assembly design. Configurable X-Ray beam collimators (both on source and detector side) and their design may be used, as well as an open collimator setup (the detector is fully exposed) for system motion registration. A narrow beam collimator setup may be used to mitigate scattering effects in order to achieve the required CT number uniformity and accuracy. Further, use of special attenuation blades mounted on the detector collimation unit in order to monitor and correct the overall acquisition gain and offset. Finally, a CT algorithm of reconstruction with integrated corrections for mitigating non-linearities of all kinds including but not limited to a scatter, beam hardening, detector saturation, lost “skin level”, detector and tube instabilities such as warming, wear and tear, after-glow.

### RELATED APPLICATION

The present disclosure claims the filing benefit of U.S. Provisional Application No. 63/371,158, titled “System and Method For Tomographic Imaging Of Core Samples” filed on Aug. 11, 2022. The '158 application is hereby incorporated by reference in its entirety.

### TECHNICAL FIELD OF THE INVENTION

The present invention relates to tomography. More specifically, the invention relates to tomographic imaging used for checking core samples.

### BACKGROUND OF THE INVENTION

The process of extracting oil from an oil-bearing reservoir has (typically, at least) a primary phase and a secondary phase. During the primary phase, reservoir drive comes from a number of natural mechanisms. These mechanisms include natural water displacing oil downward into a well; expansion of associated petroleum gas at the top of the reservoir; expansion of associated gas initially dissolved in the crude oil; and, gravity drainage resulting from the movement of oil within a reservoir from the upper to the lower parts where wells are located. Typically, underground pressure in an oil reservoir is sufficient to force oil (along with some associated gases) to the surface. As such, it is only necessary to place a complex arrangement of valves and pipes on a well-head to connect discharging flow to a pipeline network for storage and processing. The productivity factor during the primary phase is commonly in the range of 5 to 15%.

Unfortunately, over the lifetime of a well, natural pressures decrease until, at some point, there is insufficient underground pressure to force oil to the surface. That is substantially when the secondary phase begins—i.e., after the natural reservoir drive diminishes. During this phase, secondary methods are used to supply external energy to the reservoir. Secondary methods require injecting fluids downward to increase reservoir pressure, hence increasing or replacing the natural reservoir drive with an artificial drive. Secondary techniques increase the reservoir's pressure by water/solution injection, or gas reinjection. These techniques allow an increase of the productivity factor during the secondary phase of up to 50%.

Thus, one of the main problems/tasks in the oil extraction industry—if not to say the most important and critical one—is to define and apply optimal/efficient methods, factors, and materials to supplement natural forces used to bring oil to the surface. Identifying such methods and factors are application-dependent based on characteristics of the specific oil-bearing reservoir.

In the corresponding literature, the geological formation of an oil-bearing reservoir is referred to as “porous media.” Characterization of porous media is based on a set of quantitative and qualitative considerations and factors, including:
How much oil can be potentially contained in one cubic meter (m 3) of a specific geological formation?
What is the optimal substance (e.g., saline, other water-based solution, gas) for the forced replacement process?
What would be the effective pressure of the replacing substance?
How much time will the process take?
What are the characteristics of the pores themselves, such as average size, connectivity, uniformity, etc.?

The main method of determining the characteristics of porous media begins with filling a dry sample of the geological formation (such a sample is usually called a “core”) with a substance such as oil, gas, or a saline solution, then replacing a substance contained in the pores (e.g., oil) with another one (e.g., gas or saline solution). Investigating these steps facilitates determining characteristics. However, each of these steps requires a special lab infrastructure providing a flow of liquid through the core under a pressure which is typical for natural conditions (i.e., a few thousand meters below sea level).

Numerical characterization, in particular, is based on accurate measurements of the core sample density (in milligram per cubic centimeter, mg/cm 3). By measuring density of a dry sample and then a density of the same sample fully filled (i.e., saturated), it can be determined how much liquid the sample may contain. By repeating density measurements in specific small location within the core, factors of the absorption/replacement dynamics under conditions of different geological formations, applied pressure, and other relevant factors can all be determined. By taking these density measurements in various small locations of the sample, the factors related to porous (non)uniformity can be determined.

Since dependencies between a material type, its density and its X-Ray attenuation are thoroughly investigated and well-known, using computed tomography techniques for porous media characterization has been found to be a desirable methodology.

Until the invention of the present application, these and other problems in the prior art went either unnoticed or unsolved by those skilled in the art. The present invention provides methods which, with the associated device, replicate the desired natural processes and facilitate determining critical characteristics of geological formations without sacrificing accuracy.

### SUMMARY OF THE INVENTION

There is disclosed herein an improved core sample tomographic imaging system and method which avoid the disadvantages of prior CT devices while affording additional structural and operating advantages.

Generally speaking, the computer tomographic system is comprised of a robotic arm/gantry having both an X-Ray emitter and an X-Ray detector positioned thereon and configured for CT scanning of core samples.

In specific disclosed embodiments, the CT scanning system comprises an industrial robot with X-Ray source and detector assembly as a tool to perform CT scanning of geological formation samples and such assembly design. Configurable X-Ray beam collimators (both on source and detector side) and their design may be used, as well as an open collimator setup (the detector is fully exposed) for system motion registration. A narrow beam collimator setup may be used to mitigate scattering effects in order to achieve the required CT number uniformity and accuracy.

In further embodiments, use of special attenuation blades mounted on the detector collimation unit in order to monitor and correct the overall acquisition gain and offset. Finally, a CT algorithm of reconstruction with integrated corrections for mitigating non-linearities of all kinds including but not limited to a scatter, beam hardening, detector saturation, lost “skin level”, detector and tube instabilities such as warming, wear and tear, after-glow.

Methods for performing scanning and analysis of core samples using embodiments of the disclosed system, are also disclosed herein.

These and other aspects of the invention may be understood more readily from the following description and the appended drawings.

### BRIEF DESCRIPTION OF THE DRAWINGS

For the purpose of facilitating an understanding of the subject matter sought to be protected, there are illustrated in the accompanying drawings, embodiments thereof, from an inspection of which, when considered in connection with the following description, the subject matter sought to be protected, its construction and operation, and many of its advantages should be readily understood and appreciated.

FIG. 1 is a perspective view of an embodiment of the disclosed system during removal of fluid from core samples;

FIG. 2 is a close up view of the embodiment of FIG. 1 showing geological core samples positioned within a sample station;

FIG. 3 is a perspective view of the embodiment of FIG. 1 showing an embodiment of a CT device moving to access a dry geological core sample for analysis;

FIGS. 4-8 are perspective views of the embodiment of FIG. 1 showing the disclosed CT device moving to take 360 degree scans of dry geological core samples;

FIGS. 9-11 are close up views of the embodiment of FIG. 1 showing dry geological core samples being filled with fluid;

FIG. 12-17 are perspective views of the embodiment of FIG. 1 showing the disclosed CT device moving to take 360 degree scans of the filled/saturated geological core samples;

FIG. 18 is an embodiment of an X-Ray source side collimator with four X-Ray beam stopping tungsten blades controlled independently;

FIG. 19 is an embodiment of an X-Ray detector side removable collimator with X-Ray beam anti-scatter blades; and

FIG. 20 is top/side view of an embodiment of an X-ray detector side removable collimator having copper blades positioned for monitoring overall acquisition channel gain; and

FIG. 21 is a perspective view of a partial assembly of an embodiment of an X-Ray detector side removable collimator with mounting strips.

### DETAILED DESCRIPTION OF THE INVENTION

While this invention is susceptible of embodiments in many different forms, there is shown in the drawings and will herein be described in detail at least one preferred embodiment of the invention with the understanding that the present disclosure is to be considered as an exemplification of the principles of the invention and is not intended to limit the broad aspect of the invention to any of the specific embodiments illustrated.

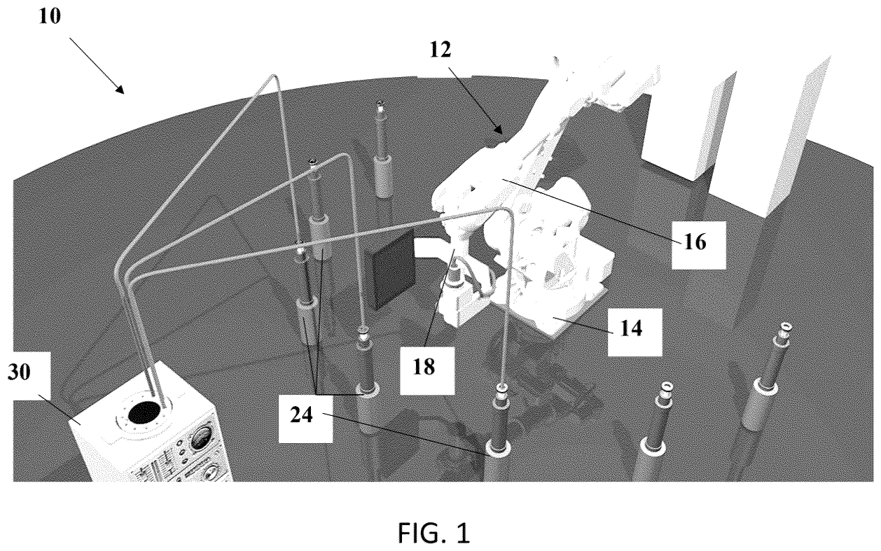

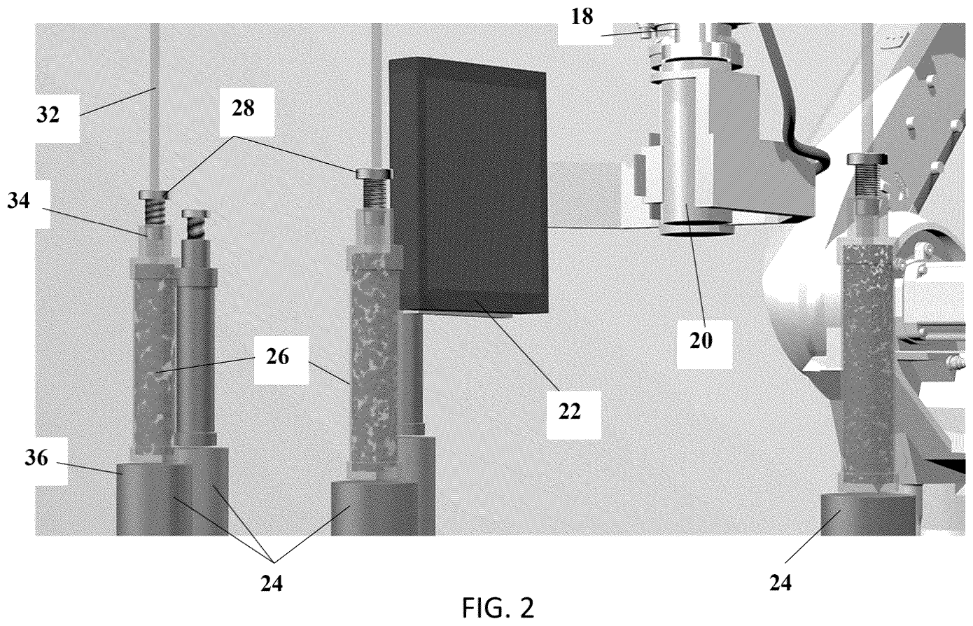

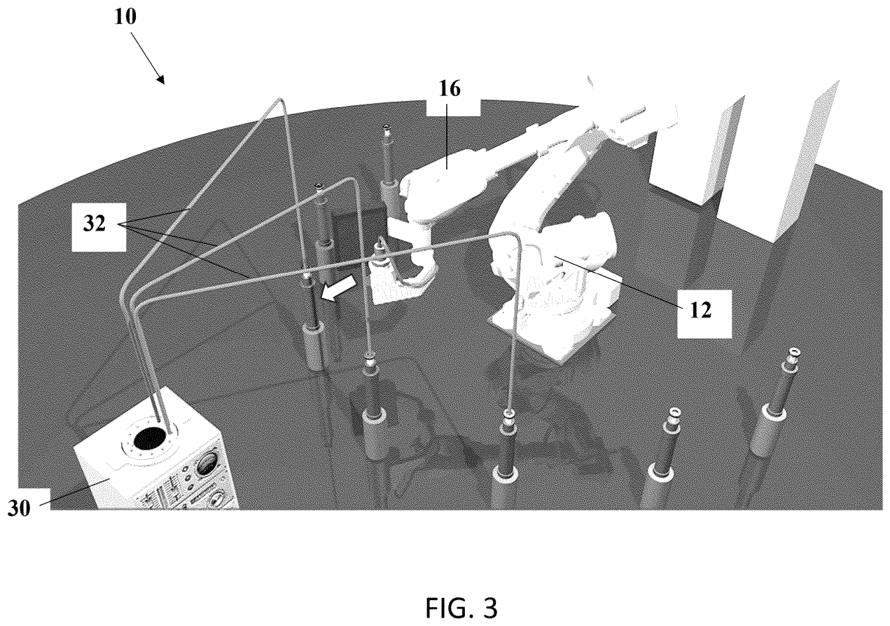

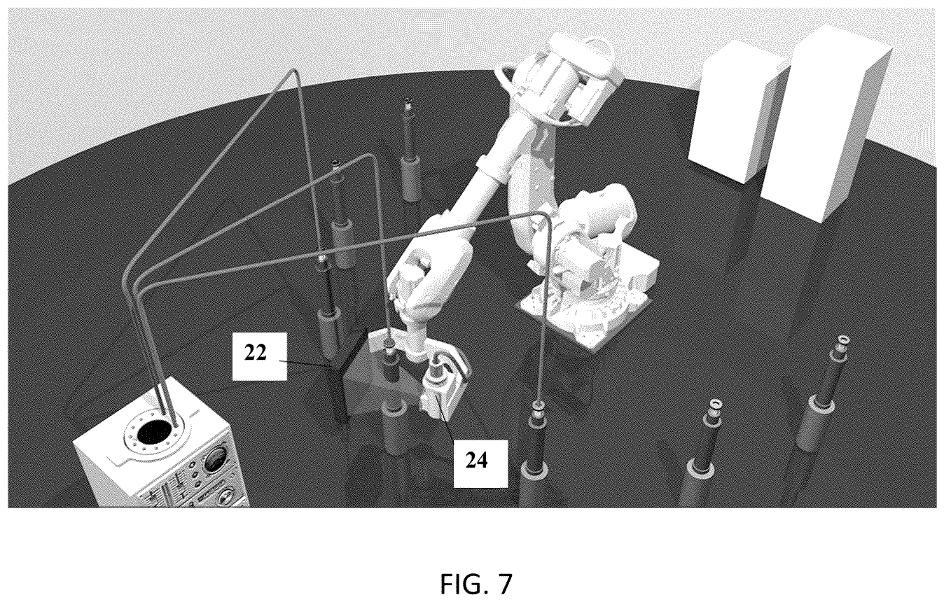

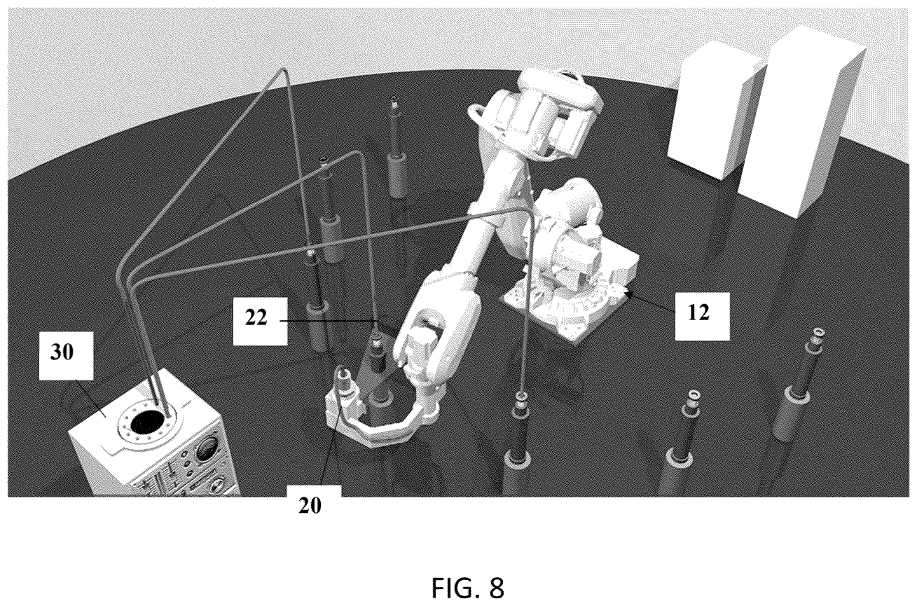

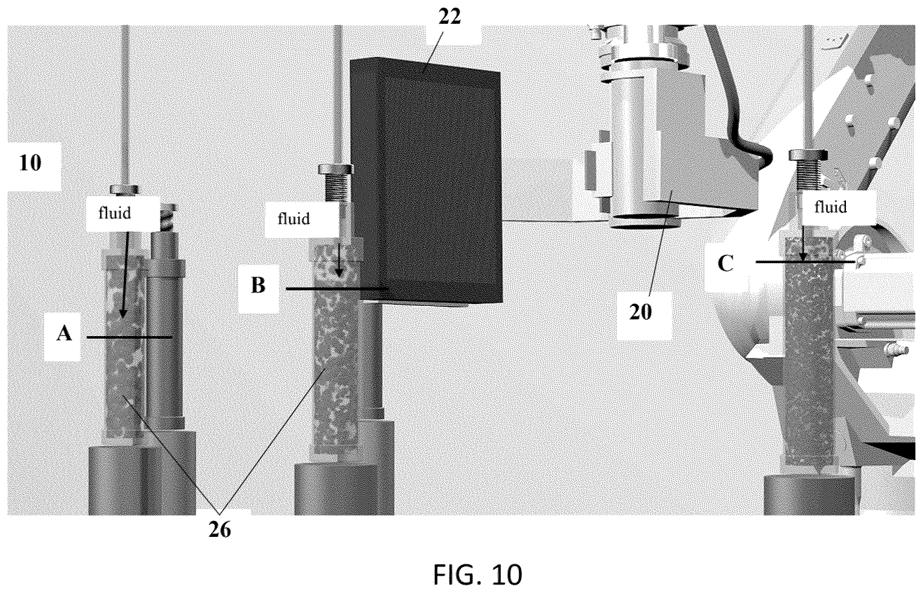

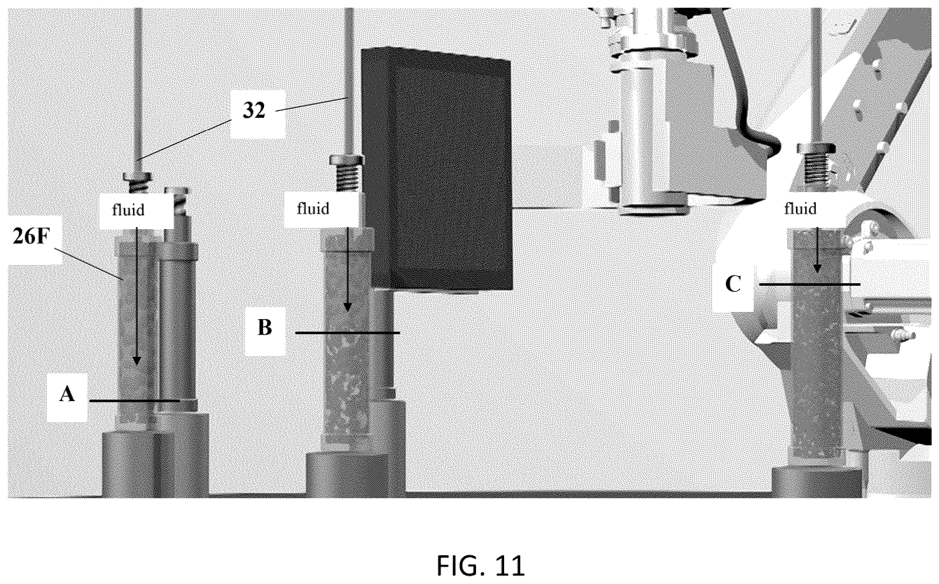

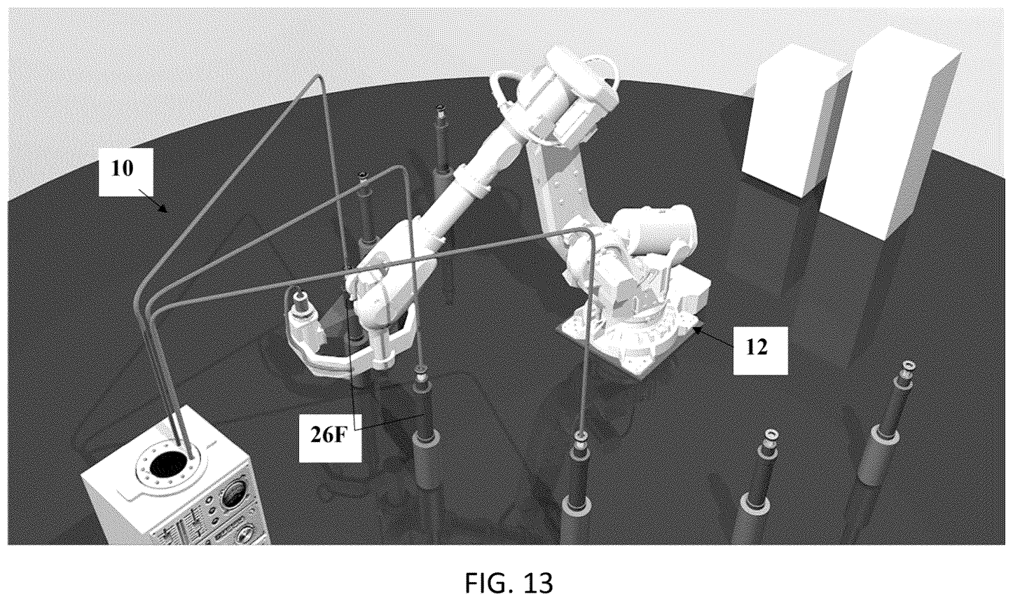

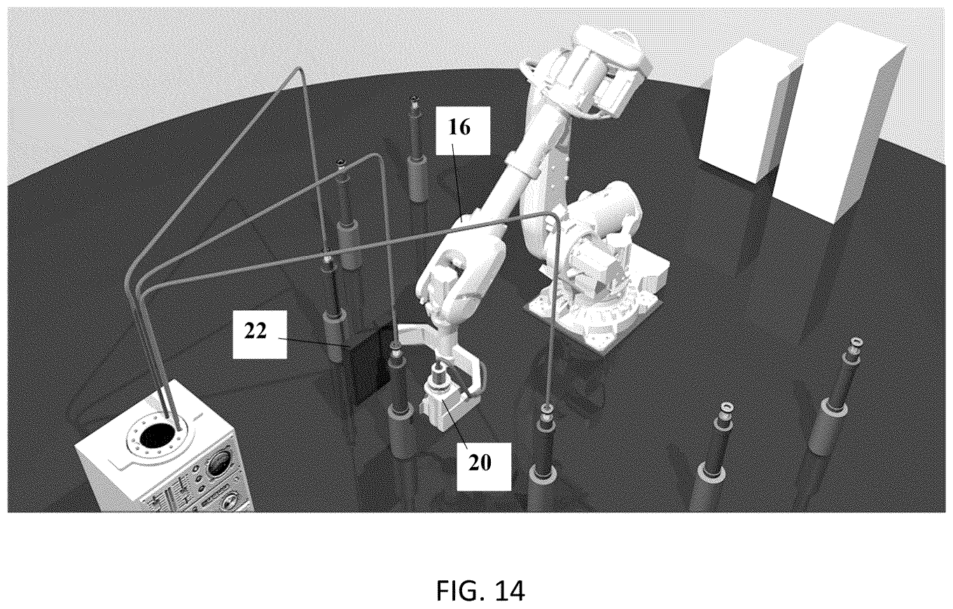

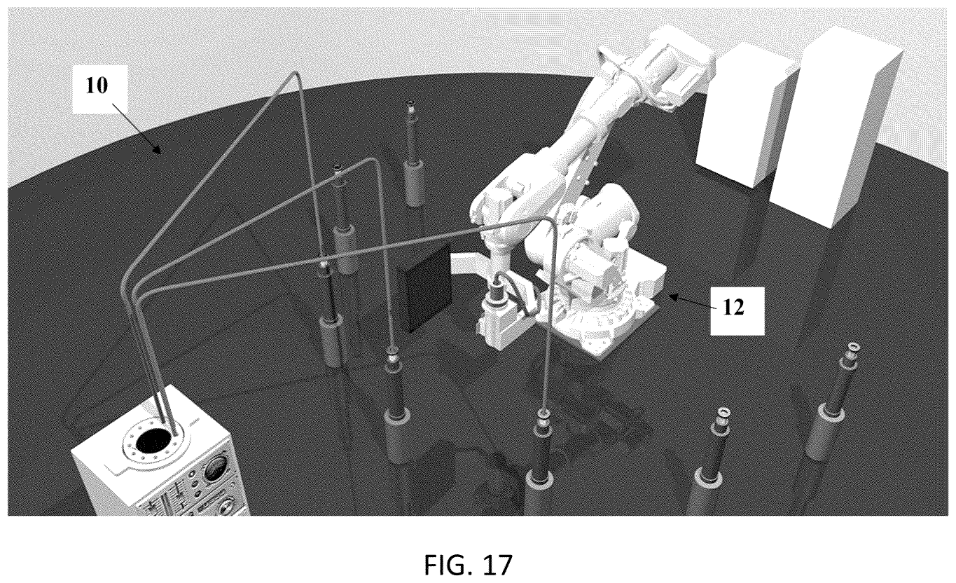

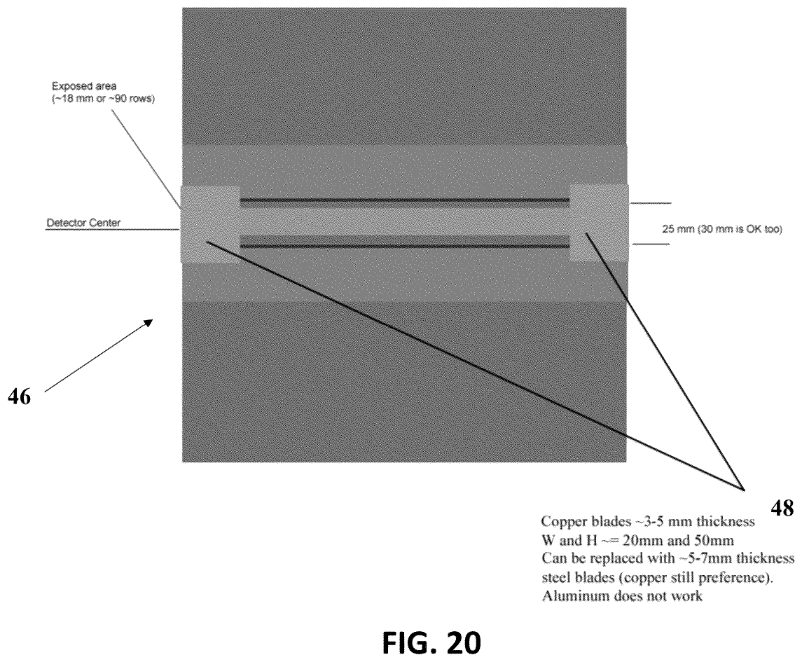

Referring to FIGS. 1-20, there is illustrated a computer tomography (CT) system for analyzing geological core samples, generally designated by the numeral 10. The particular illustrated scanning/analysis device and the corresponding methods for analyzing geological core samples are key elements for the present disclosure. However, while all the embodiments illustrated and described are directed to core sample analysis, it should be understood that the principles of the invention can be more broadly applied.

As can be seen in FIG. 1, a preferred embodiment of the system 10 is comprised of a CT device 12 having a stationary base 14, a robotic arm (or gantry) 16 extending from the base 14 with a full range of motion, and a CT scanning head 18 attached to the end of the robotic arm 16, and at least one core sample station 24. The preferred scanning head 18 of the CT device 12 comprises both an X-Ray emitter 20 (or source) and, spaced at a distance, an X-Ray detector 22. In alternate embodiments, the emitter 20 and detector 22 may be on separate arms (not shown) which move synchronously during scanning. A preferred embodiment of the CT device 12 as manufactured and sold by the Assignee, ORIMTECH, is referred to as GEDAMIS, or the Geophysics Dual-Arm Multi-Modality Imaging System (see https://orimtech.com/gedamis). Further, at least one embodiment of the CT device 10 and its operations for producing 3D images is described in U.S. Patent Application Publication No. 2021/0390217, filed Jun. 10, 2021, published Dec. 23, 2021, and titled “System and Method for Performing Spriral-Trajectory Tomosynthesis.” The '217 publication is hereby incorporated by reference.

With reference to FIGS. 1-3, a preferred embodiment of the system 10 is illustrated. Structurally, the system 10 includes a plurality of core sample stations 24 (seven are shown) in proximity to the CT device 12. Each station 24 is configured to retain a core sample 26 for CT analysis and may include additional hardware such as connectors 28, controls 30, and tubing 32, for injecting a pressurized fluid (e.g., gas or saline solution) into core samples 26. As illustrated, core samples 26 are secured in sample stations 24 with an input pipe 34 at one end and an exhaust pipe 36 at an opposite end. This set up allows for the addition and removal of fluid, as well as an increase or decrease in core sample pressure.

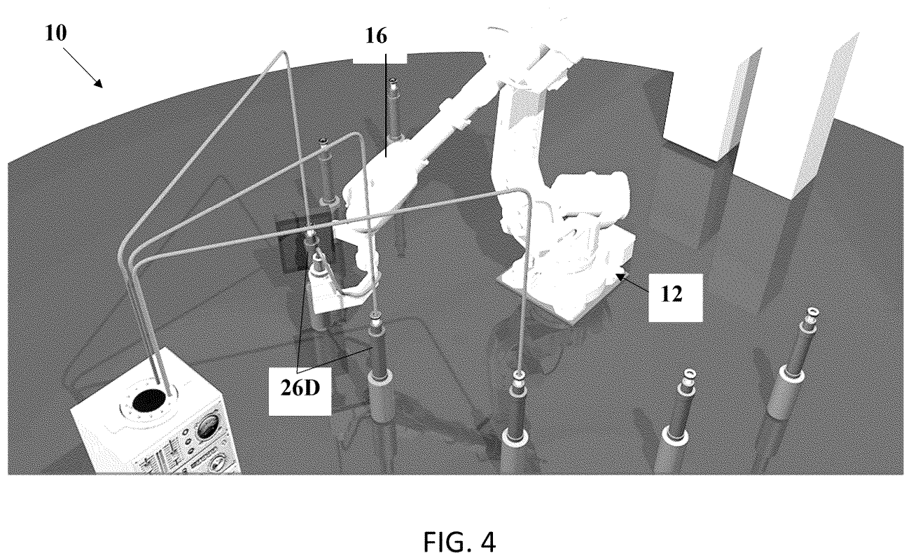

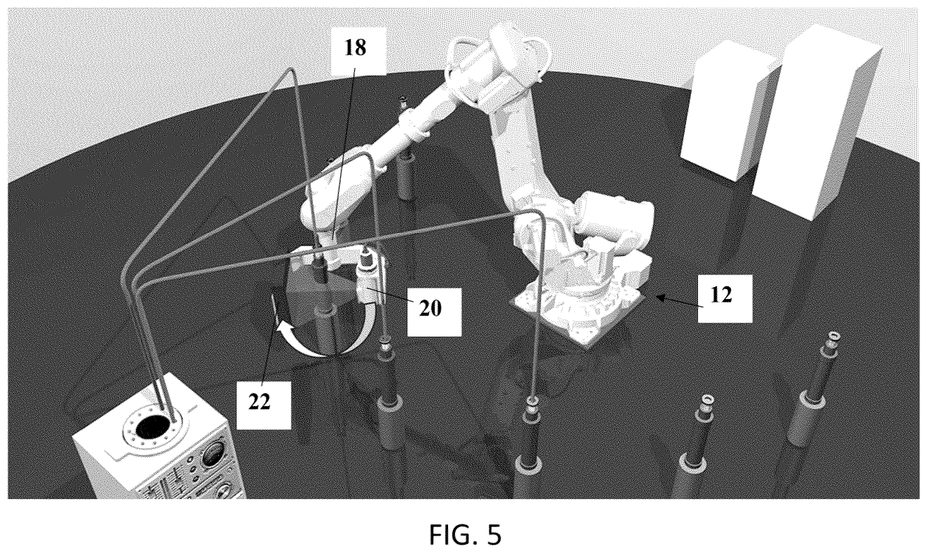

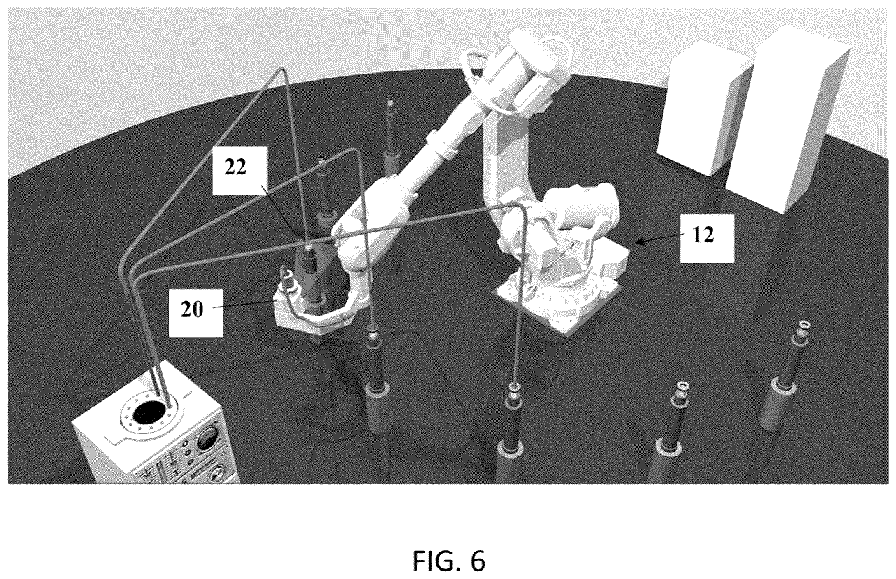

As illustrated in FIGS. 4-8, the CT device 12 is able to move, via robotic arm 16, and access core samples 26 in a manner which allows complete 360° scans for producing 3D images and calculations. Before fluid injection, the CT device 12 is able to image each of the dry core samples 26D, as best illustrated in FIGS. 5 and 6.

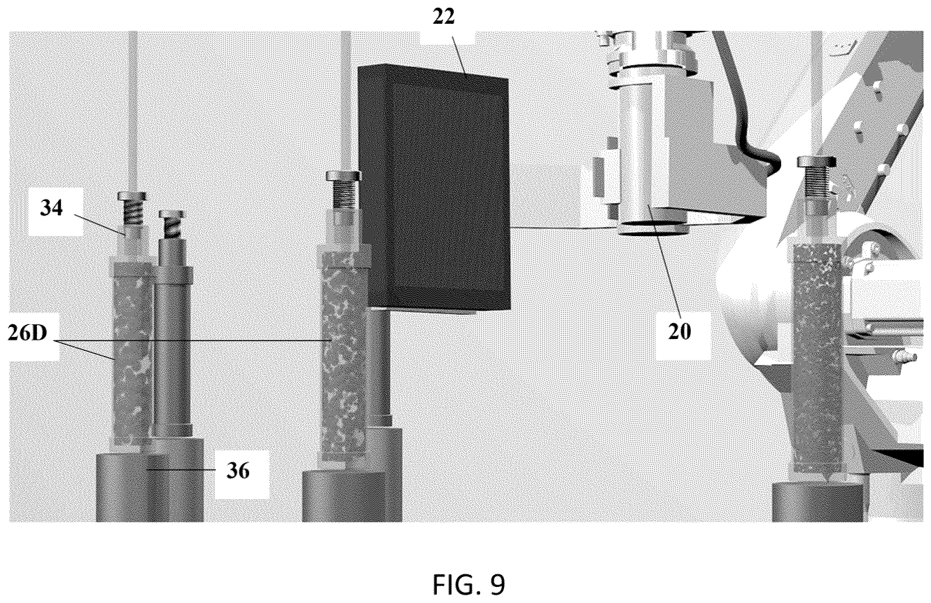

After dry analysis, the core samples can be manipulated for further measurements. For example, FIGS. 9-11 illustrate the filling of dry core samples 26D with a fluid (e.g., gas or saline solution). Fluid is forced under pressure into the core sample 26 through the upper input pipe 34 and permeates downward into the sample 26, as indicated by lines A, B, and C on each sample. An increase in pressure facilitates eventual saturation of the sample.

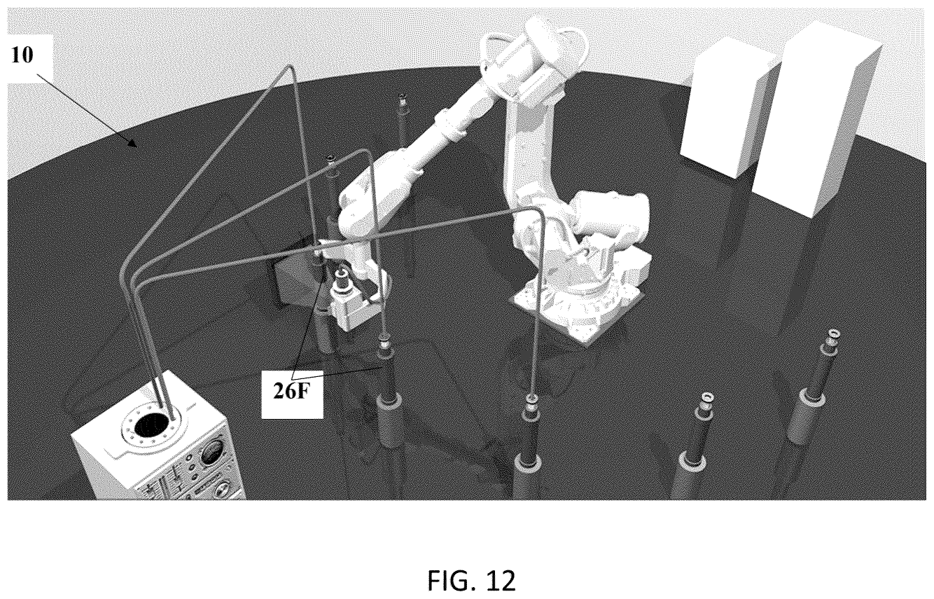

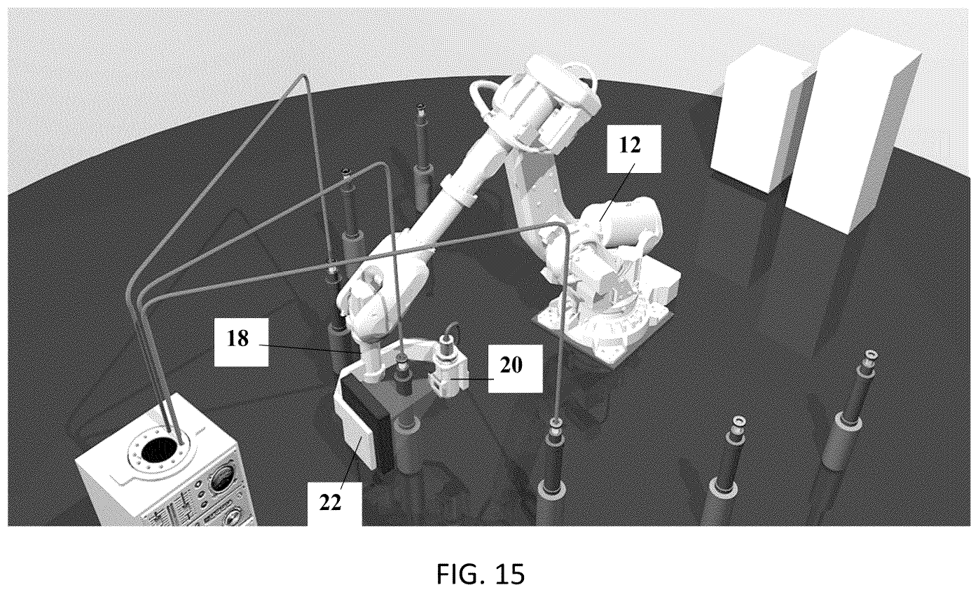

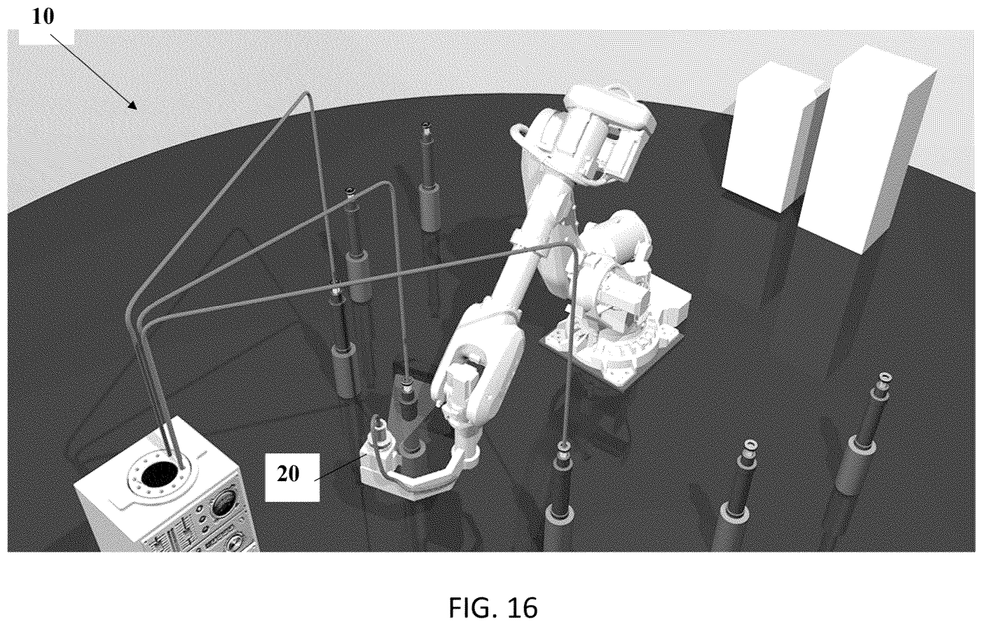

Once each of the core samples 26 has been saturated with the pressurized fluid, they can be individually re-scanned by the CT device 12. FIGS. 12-17 illustrate the plurality of once dry core samples 26D, now filled/saturated core samples 26F positioned for analysis. As before, the CT device 12 is able to access all samples 26 and take complete 360° scans for 3D images and calculations, as best shown in FIGS. 15 and 16.

As to the calculations, knowing CT numbers (values in the 3-dimensional CT image) there are direct formulas to calculate all factors mentioned above. In particular, the following values are defined:
Porosity (per cm 3)=volume of the saturating liquid contained in one cubic centimeter of sample;
Volume of the saturating liquid contained in one cubic centimeter of sample=Weight of the liquid contained in one cubic centimeter of sample divided by the liquid density (known constant);
Weight of the liquid contained in one cubic centimeter of sample=Saturated core density minus dry core density; and
Saturated core density minus dry core density=(CT number for saturated core−CT number for dry core) multiplied by attenuation constant for the used liquid (known number).

The formulas/rules above are simple, straight-forward and make CT technology very appropriate/desirable for porous media research. However, there are impediments in utilizing CT methods for such an application.

For example, conventional medical-type spiral (helical) CT devices cannot be used directly since measured samples must be part of a complex lab infrastructure. In particular, a sample has to be placed into a special core-holder, which is connected to feeding and exhausting pipes. Due to the required use of high levels of pressure and all corresponding safety requirements, such necessary hardware, is complex, massive, and very heavy. As a result, some of the additional complications include:
Filling/saturation timing, as the process typically takes a few hours;
Need of monitoring/measuring multiple samples to form data which can be considered representative for the investigated geological formation; and
Need of applying extra-high energies to penetrate core-holder and sample. It introduces a substantial non-linearity into the acquisition process due to detector over-saturation. Such non-linearity makes conventional CT reconstruction algorithms non-applicable.

Some of these impediments become irrelevant when the scanning X-Ray hardware (i.e., source 20 and areal detector 22) is mounted on industrial robot(s) as tool(s). In particular, the assembly of a gantry 16, source 20 and detector 22 are much more flexible tools for accessing a sample 26 and making a scanning motion around it. Using a pair of robots makes such a system applicable in many cases when mono-gantry does not work (e.g., due to infrastructure topology).

At the same time, the inventive scanning system 10 includes the following unique abilities:
Precise registration of system motion (precise measurements of source/detector trajectories) in 3D domain;
Strong requirements of CT number accuracy, which is contradictory to the use of areal detector (makes the case for cone-beam CT), and acquisition non-linearity.

In the described system 10 these two problems are resolved by the following:
(Re)configurable X-Ray beam collimation units allow control of the beam aperture in a wide range, from a few millimeters up to the full areal detector exposition; and
Special X-Ray attenuation blades as parts of a collimator assembly for controlling stability of X-Ray beam intensity and power, detector sensitivity in particular and the overall acquisition system gain and offset factors (also known as bright- and dark-field control).

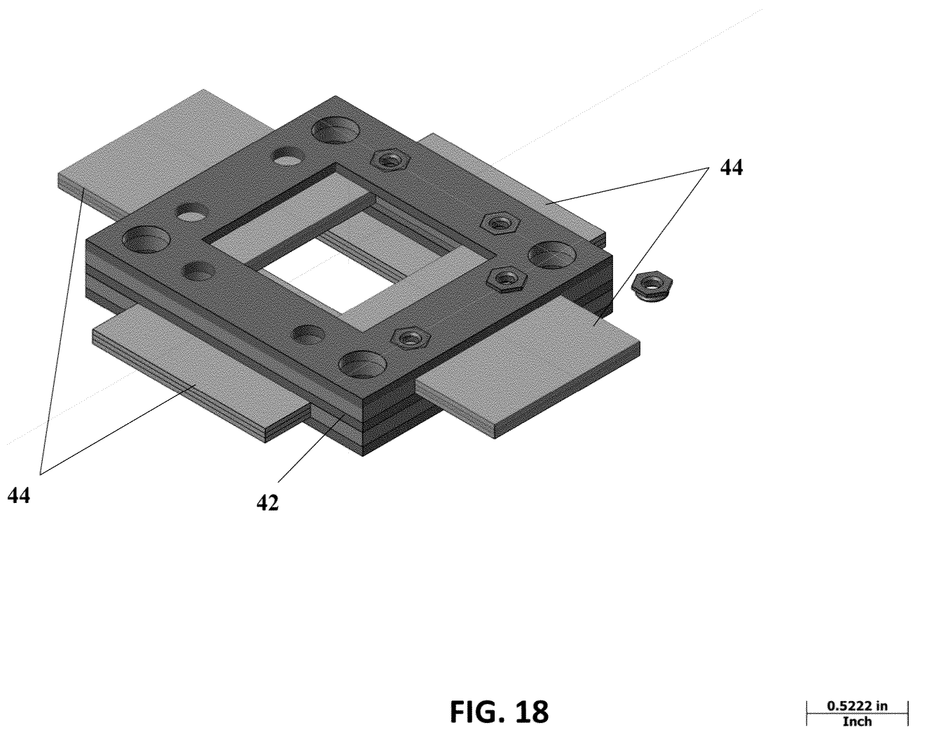

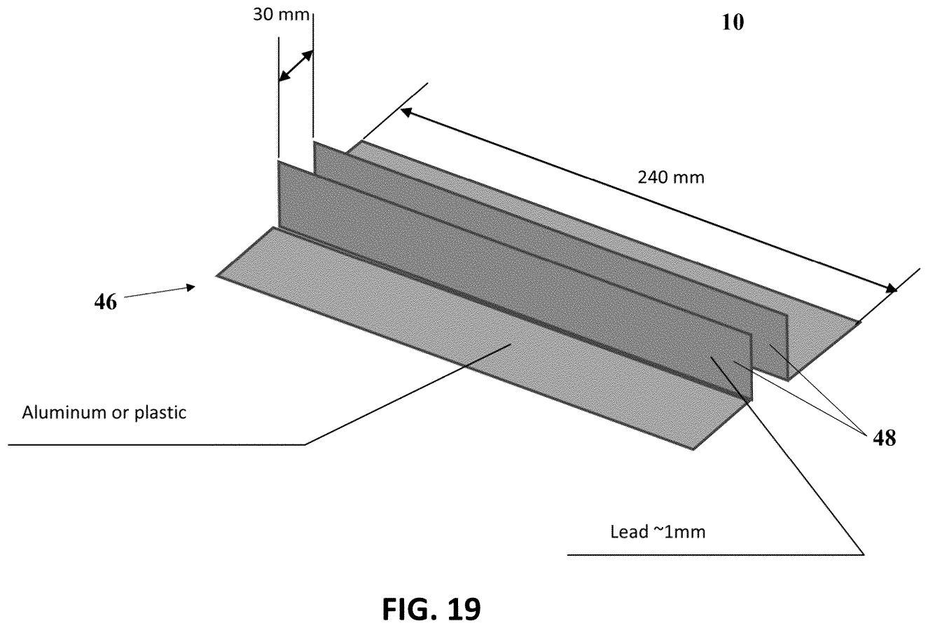

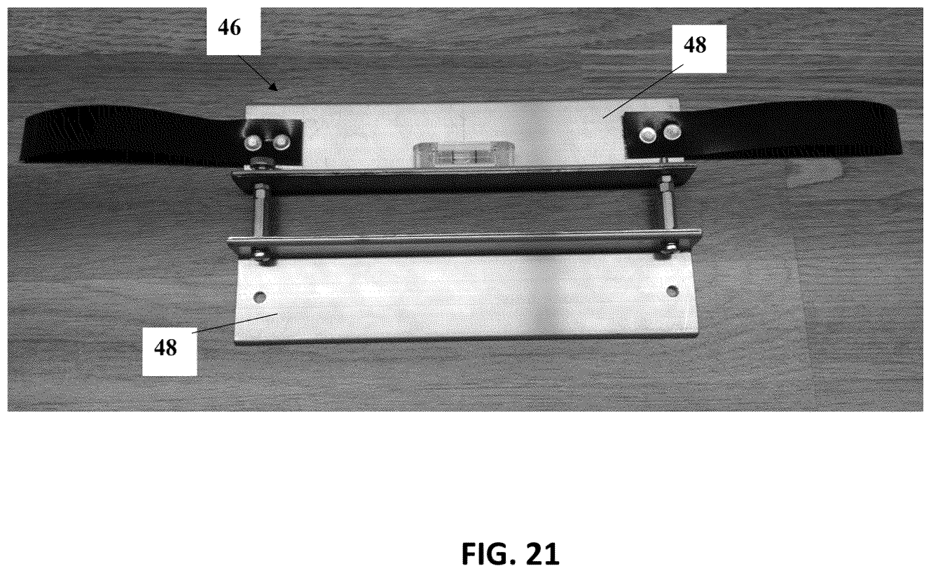

With reference to FIGS. 18-21, embodiments of preferred collimator devices 42 and 46 are illustrated. Collimator 42 (FIG. 18) is for an X-ray source side and includes four beam stopping blades 44, preferably comprised of tungsten, which are independently controlled to shape and limit an X-ray beam. FIGS. 19-21 show embodiments of removable detector side collimators 46, including a design with anti-scatter blades 48.

Accordingly, the inventive concepts disclosed include:
Use of an industrial-type (or similar) robot with X-Ray source and detector assembly as a tool with designated sample stations to perform CT scanning of geological formation core samples and such an assembly design;
Use of configurable X-Ray beam collimators (both on source and detector side) and their design;
Use of open collimator setup (the detector is fully exposed) for system motion registration (the technique and algorithm is given in U.S. Pat. No. 11,099,140 to Goldberg and assigned to Orimtech, hereby incorporated by reference);
Use of narrow beam collimator setup to mitigate scattering effects in order to achieve the required CT number uniformity and accuracy;
Use of special attenuation blades mounted on the detector collimation unit in order to monitor and correct the overall acquisition gain and offset, and the design of such blades; and
CT algorithm of reconstruction with integrated corrections for mitigating non-linearities of all kinds including but not limited to a scatter, beam hardening, detector saturation, lost “skin level”, detector and tube instabilities such as warming, wear and tear, after-glow.

The matter set forth in the foregoing description and accompanying drawings is offered by way of illustration only and not as a limitation. While particular embodiments have been shown and described, it will be apparent to those skilled in the art that changes and modifications may be made without departing from the broader aspects of applicants' contribution. The actual scope of the protection sought is intended to be defined in the following claims when viewed in their proper perspective based on the prior art.

What is claimed is:

1. A method for analyzing geological core samples comprising:
removing any fluid from at least one geological core sample to produce at least one dry geological core sample;
positioning the at least one dry geological core sample in a sample station;
conducting a first 360 degree scan of the at least one dry geological core sample using one of either a cone-beam or a fan-beam methodology of computed tomography (CT) in the same apparatus;
producing a 3D image of the first 360 degree scan;
determining a density of the at least one dry geological core sample;
adding a pressurized fluid to the at least one dry geological core sample to produce at least one saturated geological core sample;
conducting a second 360 degree scan of the at least one saturated geological core sample using one of either a cone-beam or a fan-beam methodology of a computed tomography (CT) in the same apparatus;
producing a 3D image of the second 360 degree scan; and
computing data representative of the geological core sample based on the first and second 360 degree scans.

2. The method of claim 1, wherein the CT apparatus comprises a robotic gantry having both an X-ray emitter and an X-ray detector.

3. The method of claim 1, determining an amount of oil which could be contained by each of the at least one geological core samples.

4. The method of claim 2, further comprising using X-Ray attenuation blades as parts of a collimator assembly for controlling stability of X-Ray beam intensity and power.

5. The method of claim 2, further comprising mitigating scattered photon contribution to 3D images.

6. The method of claim 5, wherein the step of mitigating scattered photon contribution to 3D images comprises using X-ray attenuation blades on the X-ray detector.

7. The method of claim 2, further comprising using X-Ray attenuation blades as parts of a collimator assembly for controlling stability of X-Ray beam techniques, such as energy and intensity, and for mitigation of scattered photon contribution to the acquired 3D views.

8. The method of claim 4, wherein the collimator assembly is positioned on the X-ray detector.

9. The method of claim 4, wherein the collimator assembly is positioned on the X-ray emitter.

10. A method for analyzing core samples, the method comprising:
obtaining at least one core sample from a geological formation;
removing all fluid from the at least one core sample to create at least one dry core sample;
performing a first CT scan on the at least one dry core sample;
filling the at least one dry core sample with a fluid to create at least one saturated core sample;
performing a second CT scan on the at least one saturated core sample;
using data from the first and second CT scans to determine characteristics of the at least one core sample from a geological formation including:
porosity (per cm 3), which is a volume of the fluid contained in one cubic centimeter of at least one core sample;
volume of the saturating liquid contained in one cubic centimeter of sample equals weight of the liquid contained in one cubic centimeter of sample divided by the liquid density;
weight of the liquid contained in one cubic centimeter of sample equals a saturated core density minus a dry core density; and
saturated core density minus dry core density equals CT number for saturated core minus CT number for dry core multiplied by an attenuation constant for the fluid used to fill the core sample.

11. A system for analyzing core samples comprising:
a core sampling station for positioning a geological core sample for analysis;
an industrial robotic gantry having an X-Ray source attached to a movable arm and an X-Ray detector attached to same movable arm, wherein the movable arm is configured to access the geological core sample positioned within the core sampling station;
a fluid delivery system configured to couple to the geological core sample positioned within the core sampling station and manipulate fluid into and out of the geological core sample;
wherein the X-Ray source and X-Ray detector are configured as a tool to perform 360 degree CT scanning of geological core samples.

12. The system of claim 11, further comprising at least one configurable X-Ray beam collimator on one of either the X-Ray source, the X-Ray detector, or both.

13. The system of claim 11, further comprising an open collimator setup for system motion registration.

14. The system of claim 11, further comprising a CT algorithm of reconstruction with integrated corrections for mitigating non-linearities of all kinds including but not limited to a scatter, beam hardening, detector saturation, lost “skin level”, detector and tube instabilities such as warming, wear and tear, after-glow.
